(19) United States
(12) Patent Application Publication
(10) Pub. No.: US 2022/0023493 A1
(43) Pub. Date: Jan. 27, 2022

(54) COMPOSITIONS AND METHODS FOR REGENERATION OF BONE TISSUE

(71) Applicant: RevBio, Inc., Lowell, MA (US)

(72) Inventors: George W. Kay, Sharon, MA (US); Brian J. Hess, Charlestown, MA (US)

(21) Appl. No.: 17/221,339

(22) Filed: Apr. 2, 2021

Related U.S. Application Data
(63) Continuation of application No. 15/771,831, filed on Apr. 27, 2018, now abandoned.
(60) Provisional application No. 62/247,497, filed on Oct. 28, 2015.

(51) Int. Cl. A61L 27/12; A61L 27/54; A61K 31/661; A61P 19/00; A61K 33/42

(52) U.S. Cl. CPC A61L 27/12; A61L 27/54; A61K 31/661; A61L 2300/112; A61K 33/42; A61L 2430/02; A61P 19/00

(57) ABSTRACT

Embodiments of the disclosure relate to compositions that stimulate the generation or regeneration of bone tissue, increasing the rate of bone healing or repair inducing the formation of osteoblasts, inducing the differentiation of mesenchymal stem cells, and the treatment of a disease or disorder in a subject.

### CROSS REFERENCE TO RELATED APPLICATIONS

This application is a continuation of U.S. patent application Ser. No. 15/771,831, filed Apr. 27, 2018, which is a U.S. National Stage Application under 35 U.S.C. § 371 of International Application No. PCT/US2016/059574, filed Oct. 28, 2016, which claims priority to U.S. Provisional Application 62/247,497, filed Oct. 28, 2015. The entire contents of each of the foregoing applications are incorporated herein by reference.

### FIELD

Embodiments of the disclosure relate to compositions that stimulate the regeneration of bone tissue and methods of use thereof.

### BACKGROUND

The bones of the skeleton are a metabolically active organs that undergo continuous remodeling throughout life. Bone remodeling involves the removal of mineralized bone by osteoclasts, followed by the deposition of osteoid through the action of osteoblasts, and its subsequent mineralization by deposition of calcium hydroxyapatite in the microscopic interstices of the organic osteoid matrix. The remodeling cycle consists of three consecutive phases: resorption, during which the giant, multinucleated osteoclasts degrade old bone; reversal, when mononuclear cells appear on the bone surface, and formation, when mats of osteoblasts lay down new bone until the resorbed bone is completely replaced. Bone remodeling serves to adjust bone architecture to meet changing mechanical needs and to repair micro-damage in bone matrix hence preventing its accumulation in the skeleton. The processes involved in bone metabolism and repair are complex, and involve physical-chemical influences such as the systemic acid-base balance, endocrine control at the systemic level through hormones (e.g., calcitonin, parathyroid hormone, growth hormone, corticosteroids, and others), local influences by locally-acting hormones such as the prostaglandins of the E series, and paracrine signaling at cell size distances.

In the healthy state, bone has the capacity to regenerate completely if defects are smaller than a certain critical size. The process requires a scaffold, which can consist of a fibrin network deposited as a blood clot, and can be disrupted and derailed by the presence of relative motion of the edges of the defect. When trauma, a pathological process, or a surgical procedure results in bone defects greater than the critical size, clinicians often apply bone grafts to fill them in order to restore the bone to full contour and function. The goal of bone grafting is to restore the continuity of bone through induction of the bone remodeling processes to replace the graft material with functional new bone. Bone grafting is a complex and risky surgical procedure that can pose a significant health risk to the patient, particularly when the graft fails to heal properly. As such, there is a need for new compositions and methods that may be used to regenerate bone tissue and treat or prevent the slowing of osteoblast growth in subjects in need thereof.

### SUMMARY

Described herein are compositions comprising an osteoinductive factor and methods of use thereof, including regeneration of bone tissue, induction of osteoblast formation, and treatment of a disease or disorder. In one aspect, the present disclosure features a method of regenerating bone tissue, the method comprising applying a composition comprising an osteoinductive factor to a site (e.g., into or onto bone, or in between bones). In some embodiments, the method comprises: a) preparing a composition comprising a multivalent metal salt and an osteoinductive factor in an aqueous solution or suspension; b) applying the composition to the site (e.g., into or onto bone, or in between bones); and c) allowing the composition to remain undisturbed until the composition is hardened, cured, or resorbed by bone.

In another aspect, the present disclosure features a method of inducing osteoblast formation, the method comprising applying a composition comprising an osteoinductive factor to a site (e.g., into or onto bone, or in between bones). In some embodiments, the method comprises: a) preparing a composition comprising a multivalent metal salt and an osteoinductive factor in an aqueous solution or suspension; b) applying the composition to the site (e.g., into bone, or onto or in between bone surfaces); and c) allowing the composition to remain undisturbed until the composition is hardened, cured, or resorbed by bone. In some embodiments, the formation of osteoblasts is derived from the differentiation of mesenchymal stem cells. In some embodiments, the osteoblasts increase the activity of alkaline phosphatase at a site.

In another aspect, the present disclosure features a method of increasing the rate of bone healing or bone repair in a subject, the method comprising applying a composition comprising an osteoinductive factor to a site (e.g., into or onto bone, or in between bones) of the subject. In some embodiments, the method comprises: a) preparing a composition comprising a multivalent metal salt and an osteoinductive factor in an aqueous solution or suspension; b) applying the composition to the site (e.g., into or onto bone, or in between bones) in a subject; and c) allowing the composition to remain undisturbed until the composition is hardened, cured, or resorbed by the subject. In some embodiments, the subject is suffering from a bone disease or disorder.

In another aspect, the present disclosure features a method of generating or regenerating bone tissue in a subject, the method comprising applying a composition comprising an osteoinductive factor to a site (e.g., into or onto bone, or in between bones) of the subject. In some embodiments, the method comprises: a) preparing a composition comprising a multivalent metal salt and an osteoinductive factor in an aqueous solution or suspension; b) applying the composition to the site (e.g., into or onto bone, or in between bones) in a subject; and c) allowing the composition to remain undisturbed until the composition is hardened, cured, or resorbed by the subject. In some embodiments, the subject is suffering from a bone disease or disorder. In some embodiments, the generation or regeneration of bone is derived from the increased action of osteoblast cells. In some embodiments, the osteoblasts increase the activity of alkaline phosphatase at a site.

In another aspect, the present disclosure features a method of slowing the progression of a bone disease or disorder in a subject, the method comprising applying a composition comprising an osteoinductive factor to a site (e.g., into or onto bone, or in between bones) of the subject. In some embodiments, the method comprises: a) preparing a composition comprising a multivalent metal salt and an osteoinductive factor in an aqueous solution or suspension; b) applying the composition to the site (e.g., into or onto bone, or in between bones) of the subject; and c) allowing the composition to remain undisturbed until the composition is hardened, cured, or resorbed by the subject.

In another aspect, the present disclosure features a method of treating or preventing a bone disease or disorder in a subject, the method comprising applying a composition comprising an osteoinductive factor to a site (e.g., into or onto bone, or in between bones) of the subject. In some embodiments, the method comprises: a) preparing a composition comprising a multivalent metal salt and an osteoinductive factor in an aqueous solution or suspension; b) applying the composition to a site (e.g., into or onto bone, or in between bones) of the subject; and c) allowing the composition to remain undisturbed until the composition is hardened, cured, or resorbed and replaced by bone.

In some embodiments, the bone disease or disorder comprises cancer (e.g., osteosarcoma), osteoporosis, rickets, osteogenesis imperfecta, Paget's disease of the bone, hearing loss, renal osteodystrophy, a malignancy of the bone, infection of the bone, osteonecrosis, or other genetic or developmental disease. In some embodiments, the bone disease or disorder comprises osteoporosis.

In some embodiments of any and all aspects of the present disclosure, the osteoinductive factor is a compound of Formula (I):

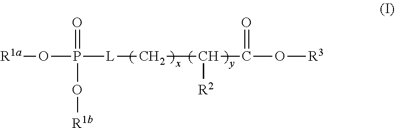

or a pharmaceutically acceptable salt thereof, wherein L is O, S, NH, or CH₂; each of R^{1a} and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3. Phosphoserine is exemplary of compounds of Formula (I).

In another aspect, the present disclosure features a method for regenerating bone tissue comprising preparation and use of a composition comprising at least two multivalent metal salts and an osteoinductive factor of Formula (I):

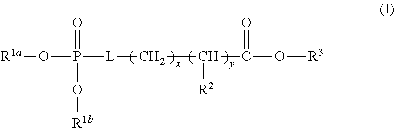

or a pharmaceutically acceptable salt thereof, wherein L is O, S, NH, or CH₂; each of Ria and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3; in an aqueous solution or suspension.

In another aspect, the present disclosure features a method of treating a subject suffering from a bone disease or disorder, the method comprising preparation and administration of a composition comprising at least two multivalent metal salts and an osteoinductive factor of Formula (I):

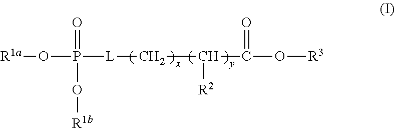

or a pharmaceutically acceptable salt thereof, wherein L is O, S, NH, or CH₂; each of Ria and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3; in an aqueous solution or suspension to thereby treat the subject.

In some embodiments, L is O or S. In some embodiments, L is O. In some embodiments, each of R^{1a} and R^{1b} is independently H. In some embodiments, L is O and each of R^{1a} and R^{1b} is H. In some embodiments, R² is H, NR^{4a}R^{4b}, or C(O)R⁵. In some embodiments, R² is NR^{4a}R^{4b}. In some embodiments, R² is NR^{4a}R^{4b} and each of R^{4a} and R^{4b} is independently H. In some embodiments, L is O, each of R^{1a} and R^{1b} is independently H, R² is NR^{4a}R^{4b}, and each of R^{4a} and R^{4b} is independently H. In some embodiments, R³ is H. In some embodiments, L is O, each of R^{1a} and R^{1b} is independently H, R² is NR^{4a}R^{4b}, each of R^{4a} and R^{4b} is independently H, and R³ is H. In some embodiments, each of x and y is independently 0 or 1. In some embodiments, each of x and y is independently 1. In some embodiments, L is O, each of R^{1a} and R^{1b} is independently H, R² is NR^{4a}R^{4b}, each of R^{4a} and R^{4b} is independently H, R³ is H, and each of x and y is 1. In some embodiments, the compound of Formula (I) is phosphoserine.

In some embodiments, the osteoinductive factor (e.g., a compound of Formula (I)) is present in an amount greater than or equal to about 10% (w/w) of the total composition. In some embodiments, the osteoinductive factor (e.g., a compound of Formula (I)) is present in an amount greater than or equal to about 1% (w/w), about 2% (w/w), about 5% (w/w), about 10% (w/w), about 11% (w/w), about 12% (w/w), about 13% (w/w), about 14% (w/w), about 15% (w/w), about 16% (w/w), about 17% (w/w), about 18% (w/w), about 19% (w/w), about 20% (w/w), about 22.5% (w/w), about 25% (w/w), about 30% (w/w), about 35% (w/w), about 40% (w/w), about 45% (w/w), about 50% (w/w), or more of the total composition.

In some embodiments, the osteoinductive factor (e.g., a compound of Formula (I)) is present in an amount greater than or equal to about 0.1% (w/w) of the composition. In some embodiments, the osteoinductive factor (e.g., a compound of Formula (I)) is present in an amount greater than or equal to about 0.1% (w/w), about 0.5% (w/w), about 1% (w/w), about 3% (w/w), about 5% (w/w), about 10% (w/w), about 20% (w/w), about 30% (w/w), about 40% (w/w), about 50% (w/w), about 60% (w/w), about 70% (w/w), about 80% (w/w), about 90% (w/w), about 95% (w/w), or up to 100% of the composition.

In some embodiments, the composition further comprises a multivalent metal salt. In some embodiments, the multivalent metal salt comprises calcium. In some embodiments, the multivalent metal salt comprises calcium and phosphate. In some embodiments, the multivalent metal salt comprises tetracalcium phosphate. In some embodiments, the multivalent metal salt comprises tricalcium phosphate. In some embodiments, the tricalcium phosphate comprises either alpha tricalcium phosphate or beta tricalcium phosphate. In some embodiments, the composition comprises a plurality of multivalent metal salts. In some embodiments, the plurality comprises tetracalcium phosphate and at least one other multivalent metal salt (e.g., a multivalent calcium compound). In some embodiments, the multivalent metal salt does not comprise tetracalcium phosphate. In some embodiments, the multivalent metal salt is present in an amount from about 15% to about 85% weight by weight (w/w) of the composition. In some embodiments, the tetracalcium phosphate is present in an amount from about 15% to about 85% weight by weight (w/w). In some embodiments, the tricalcium phosphate is present in an amount from about 15% to about 85% weight by weight (w/w).

In some embodiments, the composition comprises at least two multivalent metal salts, and at least one of the multivalent metal salts comprises an oxide. In some embodiments, at least one of the multivalent metal salts is calcium oxide. In some embodiments, the composition comprises tricalcium phosphate and calcium oxide. In some embodiments, the composition does not contain tetracalcium phosphate.

In some embodiments, the aqueous solution or suspension comprises water, saliva, saline, serum, plasma, or blood. In some embodiments, the aqueous solution or suspension comprises water. In some embodiments, the aqueous solution or suspension comprises saliva, serum or blood.

In some embodiments, the multivalent metal salt is initially provided as granules or a powder.

In some embodiments, the composition further comprises an additive.

In some embodiments, the method further comprises release of the osteoinductive factor from the composition. In some embodiments, the release of the osteoinductive factor takes place over the course of minutes, hours, days, months, or years. In some embodiments, the method further comprises release of the osteoinductive factor or the additive from the composition. In some embodiments, the release of the osteoinductive factor or the additive from the composition takes place over the course of minutes, hours, days, months, or years. In some embodiments, the additive in the composition is biologically derived (e.g., peptides, proteins (e.g., bone morphogenetic protein), or small molecules).

In some embodiments, the regeneration of bone tissue or the formation of osteoblasts is correlated with an increase in the levels of a biomarker relative to a reference standard. In some embodiments, the biomarker comprises alkaline phosphatase, osteocalcin, matrix gla protein, or osteopontin, or collagen (e.g., type I collagen).

In another aspect, the present disclosure features a kit for use in the generation or regeneration of bone tissue, wherein the kit comprises: (a) an osteoinductive factor comprising a compound of Formula (I):

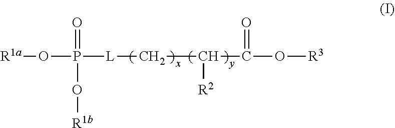

or a pharmaceutically acceptable salt thereof, wherein L is O, S, NH, or CH₂; each of Ria and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3; (b) a multivalent metal salt comprising calcium; (c) an aqueous medium; and optionally, (d) an additive (e.g., biologically active substance). In some embodiments, each of (a), (b), (c), and (d) is contained within a separate container.

In some embodiments, the kit comprises a container or plurality of containers containing a multivalent metal salt (e.g., calcium phosphates or calcium oxide) and an osteoinductive factor (e.g., phosphoserine) present together or in separate containers and sealed under good packaging practices to preserve the shelf life of the individual components. In some embodiments, the aqueous medium is water or saline.

### BRIEF DESCRIPTION OF THE DRAWINGS

FIG. 1 is a graph depicting the in vitro elution profile of phosphoserine from Composition 1 as determined by HPLC.

FIG. 2 is a graph summarizing the cell viability of osteoblasts after exposure to Composition 2 as determined by MTT assay.

FIGS. 3A-3F are photomicrographs taken of plated osteoblasts after 12 hours, 1 day, and 3 days after treatment with Composition 2 (black arrow).

FIGS. 4A-4F are photomicrographs taken of plated osteoblasts after 5 days, 7 days, and 9 days after treatment with Composition 2 (black arrow).

FIGS. 5A-5F are photomicrographs taken of plated osteoblasts after 11 days, 13 days, and 14 days after treatment with Composition 2 (black arrow).

FIG. 6 is a graph summarizing the alkaline phosphatase activity based on percent (%) of control of osteoblasts exposed to Composition 2.

FIGS. 7A-7B are photomicrographs taken at the site of the implant of Composition 2 at representative subjects at 8 weeks

FIGS. 8A-8B are photomicrographs taken at the site of the implant of Composition 2 at representative subjects at 26 weeks

FIGS. 9A-9B are photomicrographs taken at the site of the implant of Composition 2 at representative subjects at 52 weeks.

FIG. 10 is a table summarizing the semi-quantitative histological analysis collected at each of 8 weeks, 26 weeks, and 52 weeks after implantation of Composition 2.

FIGS. 11A-11C are representative photomicrographs from rabbits implanted with Composition 2 at 8 weeks (FIG. 11A), 26 weeks (FIG. 11B), and 52 weeks (FIG. 11C) after implantation.

FIGS. 12A-12H are CBCT images in the occlusal and coronal plane of the region of the maxillary fourth premolar site (#2) at immediate pre-op (FIGS. 12A and 12E), immediate post-op (FIGS. 12B and 12F), 12 weeks post-op (FIGS. 12C and 12G), and 16 weeks post-op (FIGS. 12D and 12H), relative to the implantation of Composition 3 onto the surface of bone—Canine C5.

FIG. 13 is the tissue response to Composition 3 adhered to the buccal aspect of the mandible as a subperiosteal onlay graft at ten weeks post-implantation (10×, 40×, Trichrome)—Canine C4.

### DETAILED DESCRIPTION

### Components of the Compositions

The present disclosure features compositions (e.g., adhesive compositions) comprising an osteoinductive factor and methods of use thereof, including regeneration of bone tissue, induction of osteoblast formation, and treatment of a disease or disorder. In some embodiments, the composition comprises an osteoinductive factor (e.g., a small organic phosphate). In some embodiments, the composition further comprises a multivalent metal salt (e.g., calcium phosphates, calcium oxides, and calcium hydroxide) and an osteoinductive factor (e.g., a small organic phosphate) as well as methods of use thereof. More specifically, the disclosure features methods that accelerate the rate of the healing, repair, and regeneration of bone tissue in conjunction with treatment of a bone disease or disorder, e.g., conditions related to deficiencies in volume or density of the skeletal parts or their repair, e.g., osteoporosis.

The osteoinductive factor may be described by a compound of Formula (I) or a salt thereof:

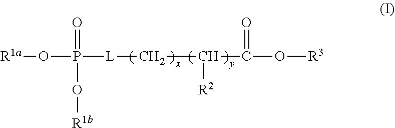

wherein L is O, S, NH, or CH₂; each of R^{1a} and R^{1b} is independently H, optionally substituted alkyl, or optionally substituted aryl; R² is H, NR^{4a}R^{4b}, C(O)R⁵, or C(O)OR⁵; R³ is H, optionally substituted alkyl, or optionally substituted aryl; each of R^{4a} and R^{4a} is independently H, C(O)R⁶, or optionally substituted alkyl; R⁵ is H, optionally substituted alkyl, or optionally substituted aryl; R⁶ is optionally substituted alkyl or optionally substituted aryl; and each of x and y is independently 0, 1, 2, or 3.

In some embodiments, L is O or S. In some embodiments, L is O. In some embodiments, each of R^{1a} and R^{1b} is independently H. In some embodiments, L is O and each of R^{1a} and R^{1b} is independently H. In some embodiments, R² is H, NR^{4a}R^{4b}, or C(O)R⁵. In some embodiments, R² is NR^{4a}R^{4b}. In some embodiments, R² is NR^{4a}R^{4b} and each of R^{4a} and R^{4b} is independently H. In some embodiments, L is O, each of R^{1a} and R^{1b} is H, R² is NR^{4a}R^{4b}, and each of R^{4a} and R^{4b} is independently H. In some embodiments, R³ is H. In some embodiments, L is O, each of R^{1a} and R^{1b} is independently H, R² is NR^{4a}R^{4b}, each of R^{4a} and R^{4b} is independently H, and R³ is H. In some embodiments, each of x and y is 0 or 1. In some embodiments, each of x and y is 1. In some embodiments, L is O, each of R^{1a} and R^{1b} is H, R² is NR^{4a}R^{4b}, each of R^{4a} and R^{4b} is independently H, R³ is H, and each of x and y is 1. In some embodiments, the osteoinductive factor (e.g., a compound of Formula (I)) is phosphoserine.

As used herein, the term “optionally substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds (e.g., alkyl, alkenyl, alkynyl, cycloalkyl, heterocycloalkyl, aryl, or heteroaryl, any of which may itself be further substituted), as well as halogen, carbonyl (e.g., aldehyde, ketone, ester, carboxyl, or formyl), thiocarbonyl (e.g., thioester, thiocarboxylate, or thioformate), amino, —N(R^{b})(R^{c}), wherein each R^{b} and R^{c} is independently H or C₁-C₆ alkyl, cyano, nitro, —SO₂N(R^{b})(R^{c}), —SOR^{d}, and S(O)₂R^{d}, wherein each R^{b}, R^{c}, and R^{d} is independently H or C₁-C₆ alkyl. Illustrative substituents include, for example, those described herein above. The permissible substituents can be one or more and the same or different for appropriate organic compounds. This disclosure is not intended to be limited in any manner by the permissible substituents of organic compounds. It will be further understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

In some embodiments, the molecular weight of the osteoinductive factor is below about 1000 g/mol. In some embodiments, the molecular weight of the osteoinductive factor is between about 150 g/mol and about 1000 g/mol, e.g., between about 155 g/mol and about 750 g/mol, between about 160 g/mol and about 500 g/mol, between about 165 g/mol and about 250 g/mol, between about 170 g/mol and about 200 g/mol, or between about 175 g/mol and about 190 g/mol. In some embodiments, the molecular weight the osteoinductive factor is between about 180 g/mol and about 190 g/mol.

The osteoinductive factor of Formula (I) may adopt any stereoisomeric form or contain a mixture of stereoisomers. For example, the osteoinductive factor may be a mixture of D,L-phosphoserine, or contain substantially pure D-phosphoserine or substantially pure L-phosphoserine. In many embodiments, the stereochemistry of the osteoinductive factor does not significantly impact the regeneration properties of the composition. In some embodiments, the particular stereochemistry of the organic phosphate or the ratio of stereoisomers of the osteoinductive factor has a significant impact on the regeneration properties of the composition.

In some embodiments, the osteoinductive factor (e.g., a compound of Formula (I)) is present in an amount greater than or equal to about 0.1% (w/w) of the composition. In some embodiments, the osteoinductive factor (e.g., a compound of Formula (I)) is present in an amount greater than or equal to about 0.1% (w/w), about 0.5% (w/w), about 1% (w/w), about 3% (w/w), about 5% (w/w), about 10% (w/w), about 20% (w/w), about 30% (w/w), about 40% (w/w), about 50% (w/w), about 60% (w/w), about 70% (w/w), about 80% (w/w), about 90% (w/w), about 95% (w/w), or up to 100% of the composition.

The compositions described herein may further comprise a multivalent metal salt. Multivalent metal salts, including calcium phosphates (e.g., tetracalcium phosphate), have been shown to react with certain phosphate-containing compounds in aqueous environments to form compositions with powerful adhesive properties. Without wishing to be bound by theory, these multivalent metal salts are thought to form ionic interactions with the phosphate-containing compounds which when combined in certain ratios react to provide a cement-like material. Exemplary multivalent metal salts may be organic or inorganic in nature and include calcium phosphates (e.g., hydroxyapatite, octacalcium phosphate, tetracalcium phosphate, tricalcium phosphate), calcium nitrate, calcium citrate, calcium carbonate, calcium sulfate, magnesium phosphates, sodium silicates, lithium phosphates, titanium phosphates, strontium phosphates, barium phosphates, zinc phosphates, calcium oxide, magnesium oxide, and combinations thereof.

The amount of each multivalent metal salt (e.g., a calcium phosphate or calcium oxide or a combination thereof) may vary, e.g., between about 10% to about 90 weight by weight (w/w) of the total composition. In some embodiments, the amount of the multivalent metal salt (e.g., a calcium phosphate or calcium oxide or a combination thereof) is in the range of about 10% to about 90%, about 15% to about 85%, about 20% to about 80%, about 30% to about 75%, about 40% to about 70%, or about 50% to about 65% w/w of the total composition. In other embodiments, the amount of the metal salt (e.g., a calcium phosphate or calcium oxide or a combination thereof) is in the range of about 5% to about 95%, about 10% to about 85%, about 15% to about 75%, about 20% to about 65%, about 25% to about 55%, or about 35% to about 50% w/w of the total composition.

In some embodiments, where the composition comprises an osteoinductive factor and a multivalent salt, the multivalent metal salt (e.g., calcium phosphates, calcium oxide or combinations thereof) reacts with an osteoinductive factor to form a composition capable of regeneration of bone tissue when combined with an aqueous medium. In some embodiments, the aqueous medium comprises water (e.g., sterile water), saliva, buffers (e.g., sodium phosphate, potassium phosphate, or saline), blood, blood-based solutions (e.g., plasma, serum, bone marrow), spinal fluid, dental pulp, cell-based solutions (e.g, solutions comprising fibroblasts, platelets, odontoblasts, stem cells (e.g., mesenchymal stem cells) histiocytes, macrophages, mast cells, or plasma cells), or combinations thereof in the form of aqueous solutions, suspensions, and colloids.

In certain embodiments, it is possible to use the components without first combining them with an aqueous medium if the composition is to be used in an environment such that the aqueous medium is already present at the site of use. In this case, the composition can be spread on, sprayed on, or otherwise applied to the site of use and combined with the aqueous medium already present at said site.

In some embodiments, the compositions may further comprise an additive. An additive may be used to impart additional functionality to the composition of the disclosure, such as improving or affecting the handling, texture, durability, strength, osteoinductive factor release, or resorption rate of the material, or to provide additional cosmetic or medical properties. Exemplary additives may include salts (e.g., sodium bicarbonate, sodium chloride, sodium phosphate, sodium hydroxide, potassium chloride), polymers, fillers or physical modifiers (e.g., granules or fibers), activity modifiers (e.g., adsorption agents), formulation bases, viscosity modifiers (e.g., polyols (e.g., glycerol, mannitol, sorbitol, trehalose, lactose, glucose, fructose, or sucrose)), bone fragments, bone chips, coloring agents (e.g., dyes or pigments), flavoring agents (e.g., sweeteners), medications that act locally (e.g., anesthetics, coagulants, clotting factors, chemotactic agents, agents inducing phenotypic change in local cells or tissues, and signaling system components or modifiers), medications that act systemically (e.g., analgesics, anticoagulants, hormones, vitamins, pain relievers, anti-inflammatory agents), antimicrobial agents (e.g., antibacterial, antiviral, or antifungal agents) or combinations thereof. The biologically active substances (e.g., medicines) in the categories above might include active substances or precursors, which become biologically active upon modification after interaction with the surrounding environment. The substances might be synthetic, semisynthetic, or biologically derived (e.g., peptides, proteins (e.g., bone morphogenetic protein), or small molecules). The substances might include, but not be limited to anti-inflammatories (e.g., steroids, nonsteroidal anti-inflammatory drugs, cyclooxygenase inhibitors), complement proteins, bone morphogenic factors and proteins, hormones active locally or systemically (e.g., parathyroid hormone, calcitocin, prostaglandins), or other small molecules (e.g., calciferols).

In some embodiments, the additive is a polymer. These polymeric based compounds may include one or more of a poly(L-lactide), poly(D,L-lactide), polyglycolide, poly(ε-caprolactone), poly(teramethylglycolic-acid), poly(dioxanone), poly(hydroxybutyrate), poly(hydroxyvalerate), poly(lactide-co-glycolide), poly(glycolide-co-trimethylene carbonate), poly(glycolide-co-caprolactone), poly(glycolide-co-dioxanone-co-trimethylene-carbonate), poly(tetramethylglycolic-acid-co-dioxanone-co-trimethylenecarbonate), poly(glycolide-co-caprolactone-co-lactide-co-trimethylene-carbonate), poly(hydroxybutyrate-co-hydroxyvalerate), poly(methylmethacrylate), poly(acrylate), a polyamine, a polyamide, a polyimidazole, poly(vinyl-pyrrolidone), collagen, silk, chitosan, hyaluronic acid, collagen, gelatin and/or mixtures thereof. In addition, co-polymers of the above homopolymers also can be used.

In some embodiments, the fillers or physical modifiers are made from tricalcium phosphate (in either the alpha or beta form), hydroxyapatite, or mixtures thereof. The fillers or physical modifiers may also be made from biodegradable polymers such as polyethylene glycol (PEG), polylactic acid (PLLA), polyglycolic acid (PGA), and copolymers of lactic and glycolic acid (PLGA), and may further comprise biodegradable block polymers such as polylactic acid (PLLA)-polyethylene glycol (PEG)-polylactic acid (PLLA) block polymer.

In some embodiments, the composition comprises a plurality of said additives. In some embodiments, certain additives may be provided as powders or granules or solutes or any combination thereof. These powders may exhibit a mean particle size of about 0.001 to about 0.250 mm, about 0.005 to about 0.150 mm, about 0.25005 to about 0.75075 mm, 0.25 to about 0.5010 to about 0.050 mm, about 0.015 to about 0.025 mm, about 0.020 to about 0.060 mm, about 0.020 to about 0.040 mm, about 0.040 to about 0.100 mm, about 0.040 to about 0.060 mm, about 0.060 to about 0.150 mm, or about 0.060 to about 0.125 mm. The mean particle size may be bi-modal to include any combination of mean particle sizes as previously described. These granules may exhibit a mean granule size of about 0.050 mm to about 5 mm, about 0.100 to about 1.500 mm, about 0.125 to 1.000 mm, 0.125 to 0.500 mm, about 0.125 to 0.250 mm, about 0.250 to 0.750 mm, about 0.250 to 0.500 mm, about 0.500 to 1.00 mm, about 0.500 to 0.750 mm. The mean granule size may be multi-modal to include any combination of mean granule sizes as previously described. In some embodiments, varying sizes of said powders or granules may be used in the adhesive composition.

In some embodiments, the composition comprises a calcium phosphate and an osteoinductive factor that react in an aqueous based medium to form a self-setting adhesive. In some embodiments, said composition is deposited by injection. In some embodiments, said composition is deposited as a powder comprising the essential components of the composition. In some embodiments, said composition is applied as a powder comprising the essential ingredients of the composition dusted, or otherwise coating, other elements of a graft. In some embodiments, the other elements might be bone chips, small bone chunks, bone blocks, other naturally-derived, semi-synthetic, or synthetic bone graft materials.

In some embodiments, the composition comprises an osteoinductive factor. In some embodiments the composition also comprises a biologically active substance as an additive. In some embodiments, said composition is applied as a solution or suspension comprising the osteoinductive factor. In some embodiments, said composition is applied as a solution or suspension comprising the osteoinductive factor and an additive. In some embodiments, said composition is applied by injection. In some embodiments, said composition is administered through a single injection (e.g., bolus) or a prolonged administration (e.g., a drip, repeated injections). In some embodiments, said composition is deposited as a powder, e.g., comprising the osteoinductive factor. In some embodiments, said composition is applied as a powder, e.g. comprising the osteoinductive factor dusted, or otherwise coating, other elements of a graft. In some embodiments, the other elements comprise bone chips, small bone chunks, bone blocks, other naturally-derived, semi-synthetic, or synthetic bone graft materials.

In some embodiments, the osteoinductive factor is released from the composition, e.g., by degradation of the composition mass or by diffusion out of the composition mass. In some embodiments, the additive is released from the composition, e.g., by degradation of the composition mass or by diffusion out of the composition mass. In some embodiments, the release of the osteoinductive factor and/or additive is controlled over a defined time interval (e.g., seconds, minutes, hours, days, or weeks). In some embodiments, the release of the osteoinductive factor and/or additive is controlled by initial concentration. In some embodiments, the release of the osteoinductive factor and/or additive is controlled by the rate of degradation of the composition mass. In some embodiments, the release of the osteoinductive factor and/or additive is controlled by the rate of diffusion of the composition mass. In some embodiments, the release of the osteoinductive factor and/or additive takes place from a device, e.g., an implantable device (e.g., implantable in the body) or a device external to the body.

### Uses of the Compositions

The compositions disclosed herein may be useful in a wide variety of applications. Exemplary uses include generation or regeneration of bone tissue, wherein the generation or regeneration of bone is derived from the increased action of osteoblast cells, wherein, the action of osteoblasts is to increase the activity of alkaline phosphatase at the site. Other exemplary uses include increasing the rate of bone healing or repair inducing the formation of osteoblasts, and inducing the differentiation of mesenchymal stem cells. Mesenchymal stem cells (“MSC”) are multi-potent adult stem cells that can be induced to differentiate into osteoblasts. Osteoblasts secrete alkaline phosphatase, osteoid and mineralize the bone matrix. The mineralized extracellular matrix is mainly composed of inorganic minerals, e.g., hydroxyapatite, but also significant amounts of type I collagen, and smaller amounts of other proteins and growth factors. The directed differentiation of MSCs can be carried out in vitro using appropriate differentiation media and can be assayed for specific markers such as presence of alkaline phosphatase (“AP”). Undifferentiated MSCs show weak AP activity, whereas the differentiated osteoblasts feature very high AP activity. Therefore, this marker is an indication of successful differentiation of MSCs into osteoblasts.

A biomarker, e.g., an extra-cellular matrix protein, can be detected and used as evidence of osteoblast differentiation. The matrix maturation phase is characterized by maximal expression of AP. At the beginning of matrix mineralization, certain proteins are expressed, such as osteocalcin (“OC”), bone sialo-protein (“BSP”), and osteopontin (“OPN”). Once mineralization is completed, calcium deposition can be visualized using appropriate staining methods. In some embodiments, analysis of a biomarker, e.g., a bone cell-specific marker such as AP, OC, and type I collagen, or detection of functional mineralization may be used to characterize osteoblasts in vitro. In some embodiments, the observation of mineralization process by osteoblasts in an in vitro culture is used as a tool for testing the effects of drug treatments and mechanical loading on bone cell differentiation and bone formation.

Traditionally, osteoconduction, osteoinduction, and osteogenesis have been used to describe various types of graft material behavior. Osteoconduction, as used herein, refers to the process of guiding the reparative growth of the natural bone through graft substance. Osteoconduction occurs when the bone graft material serves as a scaffold for new bone growth that is promoted by surrounding native bone. Osteoblasts from the margin of the defect that is being grafted utilize the bone graft material as a framework upon which to spread and generate new bone. In some embodiments, the composition disclosed herein is osteoconductive (e.g., has osteoconductive properties).

Osteoinduction, as used herein, refers to the process of regenerating new bone tissue. In some embodiments, osteoinduction involves the stimulation of undifferentiated cells to become active osteoblasts. In some embodiments, osteoinduction involves the stimulation of osteoprogenitor cells to differentiate into osteoblasts that then begin new bone formation. A bone graft material that is osteoconductive and osteoinductive will not only serve as a scaffold for currently existing osteoblasts but will also trigger the differentiation and proliferation of new osteoblasts, theoretically promoting faster integration of the graft. In some embodiments, the composition disclosed herein is osteoinductive (e.g., has osteoinductive properties). In some embodiments, the composition disclosed herein stimulates or accelerates osteoinduction in a sample or subject.

Osteogenesis is the process whereby living bone cells in the graft material contribute to bone remodeling. Osteogenesis occurs when vital osteoblasts originating from the bone graft material contribute to new bone growth. In some embodiments, the compositions disclosed herein comprise osteogenetic factors to regenerate new bone in a sample or subject.

Phosphoserine is primarily metabolized in the body by hydrolyzing enzymes, phosphatases, through cleavage of the phosphate ester bond into serine and orthophosphate ion. Phosphatases involved in the in vivo metabolism of phosphoserine include alkaline phosphatase, acid phosphatase and the phosphoserine specific enzyme phosphoserine phosphatase. Several of the phosphatases are present at the site of bone remodeling. Acid phosphatase is a product that is secreted by osteoclasts and alkaline phosphatase is a product that is secreted by osteoblasts. In some embodiments, the metabolism of phosphoserine comprised in a composition disclosed herein may be determined or monitored.

In another aspect, the present disclosure is useful in the prevention, treatment, or recovery from a disease or disorder in a subject. In some embodiments, the disease or disorder comprises a bone disease or disorder, e.g., cancer (e.g., osteosarcoma), osteoporosis, rickets, osteogenesis imperfecta, Paget's disease of the bone, hearing loss, renal osteodystrophy, a malignancy of the bone, infection of the bone, severe and handicapping malocclusion, osteonecrosis, or other genetic or developmental disease. In some embodiments, the compositions are used to regenerate bone in a defect caused by a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease. In some embodiments, a composition comprising an osteoinductive factor (e.g., as described herein) is used to stimulate or accelerate bone growth in a subject that has been weakened by a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease. In some embodiments, the subject has experienced a trauma, such as a broken bone, fractured bone, or damaged tooth relating to a disease or condition, such as cancer (e.g., osteosarcoma), osteoporosis, rickets, osteogenesis imperfecta, Paget's disease of the bone, hearing loss, renal osteodystrophy, a malignancy of the bone, infection of the bone, or other genetic or developmental disease.

The compositions and methods may be used to treat a subject suffering from or afflicted with any disease or condition that impacts the structural integrity of the bony skeleton. In some embodiments, the subject is a child. In some embodiments, the subject is an adult. In some embodiments, the subject is a senior (e.g., an adult over the age of about 50, about 55, about 60, about 65, about 70, about 75, about 80) or in a decline of the skeletal state. In some embodiments, the subject is a human or a non-human animal.

In some embodiments, the compositions and methods disclosed herein are utilized in low gravity, micro-gravity or sub-gravity conditions, e.g., as compared with the gravity conditions on Earth. In some embodiments, the diseases or disorders described herein may affect a subject differently in low gravity, microgravity or sub-gravity conditions, e.g., as compared with the gravity conditions on Earth.

In another aspect, the compositions described herein may slowly release an osteoinductive factor into the surrounding medium. In another aspect, the compositions described herein may slowly release an additive into the surrounding medium. In some embodiments, the release of the osteoinductive factor takes place over an extended period of time, e.g., seconds, minutes, hours, days, months, or years. In some embodiments, the release of the additive takes place over an extended period of time, e.g., seconds, minutes, hours, days, months, or years. In some embodiments, the composition is a material that solidifies in situ. In some embodiments, the composition is deposited as a depot for timed release of the osteoinductive factor and/or an additive. In some embodiments, the ratio of components of the composition varies depending on the disease or condition of the subject. In some embodiments, the ratio of components in the composition varies in volumetric segments.

In some embodiments, the release of the osteoinductive factor and/or additive relies on diffusion out of the depot deposit. In some embodiments, the release of the osteoinductive factor and/or additive is mediated by the degradation or resorption of the composition depot deposit. In some embodiments, the release of the osteoinductive factor and/or additive relies on modification of a device confining the osteoinductive factor and/or additive.

In some embodiments, the release of the osteoinductive factor and/or additive from the composition increases the local population of osteoblasts. In some embodiments, osteoblasts release an increase supply of alkaline phosphatase. In some embodiments, alkaline phosphatase is responsible for metabolism and release of the osteoinductive factor (e.g., phosphoserine) and/or additive from the composition. In some embodiments, this series of events repeats in an autocatalytic breakdown of the composition, which could accelerate the rate of subsequent bone formation by the local supply of osteoblasts that product osteoid. In some embodiments, the release of the osteoinductive factor and/or additive from the composition increases local deposition of bone.

In some embodiments, the rate of release of the osteoinductive factor and/or additive is affected by certain environmental conditions, e.g., ambient temperature, time of day, or gravity level. In some embodiments, the rate of release of the osteoinductive factor and/or additive under the gravity conditions of Earth is different than the rate of release of the osteoinductive factor in a micro-gravity environment.

In another aspect, the composition is applied directly to a site (e.g., into or onto bone, or in between bones) of a condition requiring bone tissue generation. In some embodiments, a condition and/or site for application of the composition comprised herein include, but are not limited to, an area of a congenital bone deficit (e.g., cleft palate or other expression of a cranio-facial anomaly), an acquired condition (e.g., osteoporosis, nephrogenic osteopathy), a traumatically induced lesion (e.g., a long bone fracture, spinal compression), a site of a pathologically induced bone lesion (e.g., site of enucleation of a cyst, granuloma, site of resection of a solid tumor, an osteonecrotic segment or dysplastic tissue), a surgical defect (e.g., site of craniotomy, odontectomy, donor site for autogenous bone graft), a site where bone growth is desired for reconstructive or cosmetic reasons (e.g., orthognathic procedures, plastic surgery for mental or malar process recontouring, spinal fusion, attachment of suture or ligature, attachment of ligament, or attachment tendon, attachment of an anchor), a site of prosthetic device attachment (e.g., hip prosthesis, dental or other endosseous implant, eposteal implant, ossicular chain reconstruction, cochlear implant, amputation stump prosthesis, calvarial plate prosthesis), a site of autogenous bone graft placement (e.g., alveolar ridge reconstruction), an area at risk of disabling consequences if a bone were to collapse because of structural weakness, and where a preventive measure is instituted (e.g., osteoporotic spine, hip, long bone, or a bone weakened by pathology such as multiple myeloma, fibrous dysplasia or another).

In some embodiments, the composition is applied in a fluid form. In some embodiments, the fluid is injected directly into or onto the target site of its planned activity. In some embodiments, the fluid is applied onto another object and then placed at the target site of its planned activity.

In some embodiments, the object onto which the composition is applied is intended for, designed for, or used for placement in the body as an implant. In some embodiments, the implant is a dental implant. In some embodiments, the implant is an orthopedic implant.

In some embodiments, the orthopedic implant is a joint prosthesis element.

In some embodiments, the orthopedic implant is an intramedullary element.

In some embodiments, the orthopedic implant is transdermal implant.

In some embodiments, the implant is transmucosal implant.

In some embodiments, the composition might be applied as a putty. In some embodiments, the composition is applied as a solid. In some embodiments, the solid is a formed or pre-formed object. In some embodiments, the formed or pre-formed object is an intramedullary insert. In some embodiments, the solid is in form of a coating on another object. In some embodiments, the other object onto which the composition is applied is intended for, designed for, or used for placement in the body as an implant. In some embodiments, the other object is a dental implant. In some embodiments, the other object is an orthopedic implant. In some embodiments, the other object is an element of a joint prosthesis.

In some embodiments, the other object is an element of a limb prosthesis.

In some embodiments, the composition is deposited confined by a device. In some embodiments, the device defines the rate of release of the osteoinductive factor and/or additive. In some embodiments, the device comprises metallic material. In some embodiments, the device comprises glassy material. In some embodiments, the device comprises plastic material. In some embodiments, the device comprises material which does not persist indefinitely in the body (e.g., in the connective tissue compartment).

In some embodiments, the device comprises material which is resorbable in the body (e.g., connective tissue compartment). In some embodiments, the device comprises material which is soluble in the body (e.g., connective tissue compartment). In some embodiments, the device comprises material which is degradable (e.g., a hydrogel, scaffold, sponge, micelle, exosome) in the body (e.g., connective tissue compartment). In some embodiments, the device comprises a removable barrier. In some embodiments, the device comprises a programmable feature that controls rate of release of the osteoinductive factor and/or additive. In some embodiments, the programmable feature is programmed before, during or after implementation.

In some embodiments, the composition might be deposited into the medullary space of the bone. In some embodiments, the composition might be deposited onto the external surface of the bone. In some embodiments, the composition might be applied to fractured or cut bone. In some embodiments, the composition might be applied to bone fragments. In some embodiments, the composition might be deposited to a site distant from the skeleton.

In another aspect, the compositions and methods disclosed herein are used to target the entire skeleton of the subject. In some embodiments, the subject is suffering from a bone disease or disorder as disclosed herein, e.g., osteopenia or osteoporosis. In some embodiments, the osteoinductive factor is introduced locally. In some embodiments, the osteoinductive factor and additive are introduced locally. In some embodiments, the osteoinductive factor is introduced (e.g., introduced systemically) as a therapeutic agent, e.g., to shift the balance in the bone metabolism toward deposition of new bone. In some embodiments, the osteoinductive factor and additive are introduced (e.g., introduced systemically) as a therapeutic agent, e.g., to shift the balance in the bone metabolism toward deposition of new bone.

In some embodiments, the osteoinductive factor is administered as a bolus. In some embodiments, the osteoinductive factor and additive are administered as a bolus. In some embodiments, the osteoinductive factor is administered at a constant rate over time. In some embodiments, the osteoinductive factor and additive are administered at a constant rate over time. In some embodiments, the osteoinductive factor is administered in repeated dosages. In some embodiments, the osteoinductive factor and additive are administered in repeated dosages.

In another aspect, stem cells (e.g., mesenchymal stem cells) are exposed to the composition disclosed herein or a component thereof (e.g., the osteoinductive factor or the multivalent metal salt) to regenerate bone in an in vitro setting. In some embodiments, the regenerated bone cells are introduced to the site requiring bone regeneration locally or systemically.

In another aspect, different variants of the components of the compositions disclosed herein may be packaged and marketed as a kit for specific indications. In some embodiments, the kit comprises a container containing an osteoinductive factor (e.g., phosphoserine). In some embodiments, the kit comprises a container containing an osteoinductive factor (e.g., phosphoserine) and an additive (e.g., biologically active substance). In some embodiments, the kit comprises a container containing a multivalent metal salt (e.g., calcium phosphates or calcium oxide). In some embodiments, the kit comprises a container or plurality of containers containing a multivalent metal salt (e.g., calcium phosphates or calcium oxide) and an osteoinductive factor (e.g., phosphoserine) present together or in separate containers and sealed under good packaging practices to preserve the shelf life of the individual components. In some embodiments, the kit comprises a container or plurality of containers containing a multivalent metal salt (e.g., calcium phosphates or calcium oxide) an osteoinductive factor (e.g., phosphoserine), and an additive (e.g., biologically active factor) present together or in separate containers and sealed under good packaging practices to preserve the shelf life of the individual components. If additives are included in said kit, they may be packaged within this container or within a separate container. The aqueous medium (e.g., solution or suspension), if included, may be provided in a separate container, or may be mixed with an osteoinductive factor and/or additive. The kit may include additional components for the preparation or application of the compositions, such as mixing bowls or surfaces, stirring sticks, spatulas, syringes, heat guns, or other preparation or delivery devices.

In some embodiments, the compositions may adopt a liquid, viscous, or pliable working state after mixing with an aqueous solution or suspension prior to hardening or curing, which is present for up to about 30 minutes or less, depending on the components of said compositions. In some embodiments, the compositions may adopt a pliable working state for less than or equal to about 30 minutes after mixing with an aqueous solution or suspension, e.g., less than about 20 minutes, less than about 15 minutes, less than about 10 minutes, less than about 5 minutes, less than about 3 minutes, less than about 2 minutes, less than about 1 minute, less than about 30 seconds, less than about 5 seconds after mixing with an aqueous solution or suspension.

In some embodiments, after a set amount of time, the compositions may adopt a hard, cement-like state. This process of conversion from the pliable working state to the cement-like state may be referred to as “hardening” or “curing.” In some embodiments, the compositions may exhibit an adhesive strength in the cement-like state in the range of about 100 KPa to about 12,000 KPa, depending on the application and the particular components and ratios of components in said adhesive compositions. In some embodiments, the adhesive strength of the compositions in the cement-like state is between about 100 KPa and e.g., about 10,000 KPa, about 9,000 KPa, about 8,000 KPa, about 7,000 KPa, about 6,000 KPa, about 5,000 KPa, about 4,000 KPa, about 3,000 KPa, about 2,000 KPa, about 1,000 KPa, about 750 KPa, about 500 KPa, about 250 KPa, or about 200 KPa. In some embodiments, the adhesive strength of the compositions in the cement-like state is between about 100 KPa, about 200 KPa, about 300 KPa, about 400 KPa, about 500 KPa, about 600 KPa, about 700 KPa, about 800 KPa, about 900 KPa, about 1,000 KPa, about 2,500 KPa, about 5,000 KPa, about 7,500 KPa, about 10,000 KPa or about 12,000 KPa. In some embodiments, the adhesive strength of the compositions in the cement-like state is in the range of about 200 KPa and about 2,500 KPa.

In some embodiments, the particular components of the compositions may be selected to achieve the desired strength depending on the intended use of the compositions. In all embodiments, a skilled practitioner (e.g., a doctor, dentist, surgeon, nurse, or other suitable person) may alter the specific components to achieve the desired adhesive properties of said composition based on the intended use or desired outcome.

In some embodiments, the composition does not comprise a multivalent metal salt. In some embodiments, the composition adopts a liquid, viscous, or pliable state after mixing with an aqueous solution or suspension which does not harden. In some embodiments, the physical state of the composition remains in a liquid, viscous or pliable state after mixing with an aqueous solution or suspension for a time period (e.g., seconds, minutes, hours, days, years) or indefinitely. In some embodiments, the composition adopts a liquid, viscous, or pliable state after mixing with an aqueous solution or suspension which cures to form a hydrogel or colloid within a time period (e.g., seconds, minutes).

### EXAMPLES

Some embodiments are further described in detail by reference to the following examples. These examples are provided for purposes of illustration and are not intended to be limiting unless otherwise specified. The disclosure should be construed to encompass any and all variations which become evident as a result of the teaching provided herein.

Without further description, it is believed that one of ordinary skill in the art can, using the preceding description and the following illustrative examples, make and utilize the compounds and practice the claimed methods. The following examples specifically point out various aspects of the disclosure, and are not to be construed as limiting in any way the remainder of the disclosure.

### Example 1: Determination of Release Kinetics of an Osteoinduction Factor from an Exemplary Composition

An exemplary composition of the disclosure was prepared and analyzed as outlined below in order to gauge the amount of phosphoserine (i.e., O-phospho-L-serine, OPLS) released from a self-setting adhesive composition during and after curing. Composition 1 consisted of 2740 mg total comprising the following components:

Tetra Calcium Phosphate, mean particle size=15-20 um, 400 mg

O-Phospho-L-Serine, OPLS: 185 mg

β-TCP granules, granule 0=0.5-1 mm; 70% porosity, pore 0=50-130 um, 100 mg

The powder blend was mixed with 133 uL sterile H₂O in a 20 mL vial. Several portions of the synthetic bone adhesive powders were individually weighed into glass vials, loaded into a freeze dryer, a vacuum pulled, back filled under N₂, and sealed with rubber stoppers. The plastic caps were crimped and the vials were gamma irradiated (15-25 kGy) prior to testing.

### Test Procedure

1. For each time point, a vial containing fully formulated and sterilized powder was emptied into a mortar and 133 uL of sterile water was added. The mixture was mixed vigorously with a pestle for 20 sec using a mixing rotation that was then reversed for the last 5 sec to create putty like consistency.
2. The putty was transferred from the mortar into a vial, which was tared on a balance. The weight of the putty was noted.
3. After two minutes from the start of mixing, a volume of sterile water equivalent to 1 mL per 100 μg of putty was added to the vial with no further mixing. The vial was then capped and left at room temperature for a specified period of time.
4. The supernatant was sampled by swirling the vial for 30 seconds and then transferring 1 mL into an appropriately labeled microfuge tube with a micropipette. The sample was refrigerated until analysis.
5. The derivatization reaction was performed in reduced lighting conditions as follows: 50 μL of sample or standard was mixed vigorously with 100 μL of a 1% solution of N-(2,4-dinitro-5-fluoro-phenyl)-1-alaninamide (FDAA or Marfey's reagent) in 50:50 acetone:acetonitrile in a glass vial. 40 μL of 0.3M sodium bicarbonate was then added and the solution was again mixed. The vials containing the reaction mixture were then capped and placed at 50° C. for 90 minutes.
6. At the end of the incubation the vials were cooled to room temperature and 10 μL of 2M HCl were added with mixing.
7. The reaction mixture was then transferred to an HPLC vial for analysis.
8. The sampled was injected onto a Zorbax SB-C18 column connected to a Waters Alliance 2695 HPLC instrument using 0.025M tri-ethyl ammonium phosphate (TEAP) A and 100% acetonitrile B at a flow of 1 mL/min.

### Elution Gradient Profile

| Time | % A | % B |
|---|---|---|
| 0 | 90 | 10 |
| 30.1 | 70 | 30 |
| 30.01 | 50 | 50 |

9. Elution from the HPLC was monitored at 340 nm.

| Time | % A | % B |
|---|---|---|
| 35 | 50 | 50 |
| 35.01 | 90 | 10 |
| 40 | 90 | 10 |

### Results

Each time point was analyzed in triplicate. The assayed time points show an OPLS level of 1.14 mg/mL after 10 min. The value climbed by 0.39 mg/mL to a maximum of 1.53 mg/mL at 8 hrs and then declined to 0.59 mg/mL by 24 hrs with a further reduction to 0.49 mg/mL at 168 hrs.

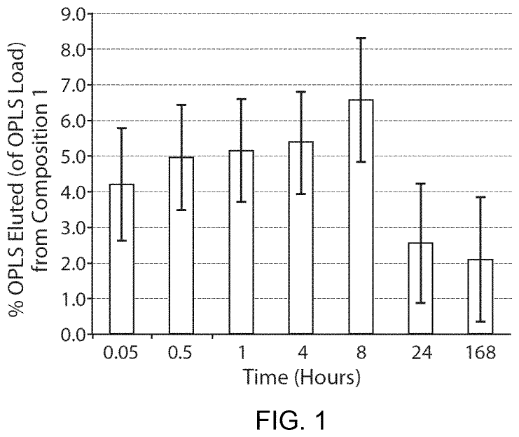

Results of the HPLC analysis are summarized in FIG. 1; Tables 1-3 below present the data collected from each of the experiments run in triplicate. It can be observed that the maximum percentage of OPLS that eluted with respect to the original formulation mass of OPLS is 7.21% at 8 hours after curing.

The data below provides baseline information only and the actual elution value may be subject to change given variations in the in vivo conditions and/or relative weight fraction of the composition components.

TABLE 1

| | | % of | | Elution | | | | |
|---|---|---|---|---|---|---|---|---|
| | mg | OPLS | mg of | Vol | Theoretical | | Measured | |
| Time | weighed | mass | OPLS | (RT | OPLS | Measured | OPLS | % of |
| point | formulation | in | in | Water), | concn, | Result, | Weight, | OPLS |
| hr | mass | pellet | pellet | ml | mg/mL | mg/ml | mg | Eluted |
| 0.05 | 515 | 62.80 | 116.19 | 5.2 | 22.34 | 1.31 | 6.81 | 5.85 |
| 0.5 | 499 | 60.85 | 112.58 | 5 | 22.52 | 1.16 | 5.80 | 5.16 |
| 1 | 443 | 54.02 | 99.95 | 4.4 | 22.71 | 1.23 | 5.41 | 5.42 |
| 4 | 581 | 70.85 | 131.08 | 5.8 | 22.60 | 0.95 | 5.51 | 4.18 |
| 8 | 620 | 75.61 | 139.88 | 6.2 | 22.56 | 1.49 | 9.24 | 6.61 |
| 24 | 565 | 68.90 | 127.47 | 5.8 | 21.98 | 0.53 | 3.07 | 2.40 |
| 168 | 499 | 60.85 | 112.58 | 5 | 22.52 | 0.52 | 2.60 | 2.29 |

TABLE 2

| | | % of | | Elution | | | | |
|---|---|---|---|---|---|---|---|---|
| | mg | OPLS | mg of | Vol | Theoretical | | Measured | |
| Time | weighed | mass | OPLS | (RT | OPLS | Measured | OPLS | % of |
| point | formulation | in | in | Water), | concn, | Result, | Weight, | OPLS |
| hr | mass | pellet | pellet | ml | mg/mL | mg/ml | mg | Eluted |
| 0.05 | 502 | 61.22 | 113.26 | 5 | 22.65 | 0.95 | 4.75 | 4.20 |
| 0.5 | 469 | 57.20 | 105.81 | 4.7 | 22.51 | 1.09 | 5.12 | 4.83 |
| 1 | 468 | 57.07 | 105.59 | 4.7 | 22.46 | 1.08 | 5.08 | 4.83 |
| 4 | 549 | 66.95 | 123.86 | 5.5 | 22.52 | 1.15 | 6.33 | 5.12 |
| 8 | 546 | 66.59 | 123.18 | 5.5 | 22.40 | 1.61 | 8.86 | 7.21 |
| 24 | 548 | 66.83 | 123.63 | 5.5 | 22.48 | 0.63 | 3.47 | 2.81 |
| 168 | 549 | 66.95 | 123.86 | 5.5 | 22.52 | 0.44 | 2.42 | 1.94 |

TABLE 3

| | | % of | | Elution | | | | |
|---|---|---|---|---|---|---|---|---|
| | mg | OPLS | mg of | Vol | Theoretical | | Measured | |
| Time | weighed | mass | OPLS | (RT | OPLS | Measured | OPLS | % of |
| point | formulation | in | in | Water), | concn, | Result, | Weight, | OPLS |
| (hr | mass | pellet | pellet | ml | mg/mL | mg/ml | mg | Eluted |
| 0.05 | 517 | 63.05 | 116.64 | 5.2 | 22.43 | 1.05 | 5.46 | 4.67 |
| 0.5 | 497 | 60.61 | 112.13 | 5 | 22.43 | 1.09 | 5.45 | 4.86 |
| 1 | 457 | 55.73 | 103.10 | 4.6 | 22.41 | 1.17 | 5.38 | 5.23 |
| 4 | 480 | 58.54 | 108.29 | 4.8 | 22.56 | 1.54 | 7.39 | 6.83 |
| 8 | 602 | 73.41 | 135.82 | 6 | 22.64 | 1.33 | 7.98 | 5.88 |
| 24 | 489 | 59.63 | 110.32 | 4.9 | 22.51 | 0.55 | 2.70 | 2.46 |
| 168 | 530 | 64.63 | 119.57 | 5.3 | 22.56 | 0.47 | 2.49 | 2.10 |

The quantities of each of the components listed may be altered or adjusted in relation to the other components in the composition. After mixing, the compositions described may be applied to the desired site and the adhesive properties examined, e.g., for tensile strength and durability.

### Example 2: Assessing the In Vitro Biocompatibility of an Exemplary Composition

The objective of this study was to determine the biocompatibility of an exemplary composition using an in vitro primary human osteoblast cell model. Composition 2 was prepared by combining 250 mg O-phospho-L-serine and 400 mg tetracalcium phosphate in 133 uL H₂O. After preparation, the composition was formed into beads and cured in 0.9% saline. Beads of the composition were rinsed with DPBS, cut to fit the wells of a 24 well plate, and individually weighed. Changes in pH were monitored at multiple times over a 24 hour period, then daily thereafter for 14 days (see Table 4). Results (mean SEM) for each control and test sample are summarized in Table 4.

TABLE 4
Localized pH changes around Composition 2 curing in 0.9% saline.

| | Control | | | |
|---|---|---|---|---|
| Time Point | (0.9% saline) | SEM | Composition 2 | SEM |
| 15 min | 6.91 | 0.003 | 4.94 | 0.012 |
| 30 min | 6.93 | 0.005 | 5.08 | 0.009 |
| 1 hour | 6.90 | 0.007 | 5.10 | 0.010 |
| 2 hour | 6.86 | 0.010 | 5.11 | 0.014 |
| 4 hour | 6.86 | 0.003 | 5.13 | 0.012 |
| 24 hour | 6.72 | 0.010 | 5.37 | 0.031 |
| (before media | | | | |
| change) | | | | |
| 24 hour (30 | 6.92 | 0.017 | 5.90 | 0.023 |
| minutes after | | | | |
| media change) | | | | |
| Day 2 | 6.85 | 0.018 | 6.07 | 0.010 |
| Day 3 | 6.69 | 0.007 | 6.06 | 0.007 |
| Day 4 (before | 6.71 | 0.009 | 6.33 | 0.018 |
| media change) | | | | |
| Day 4 (30 | 6.93 | 0.009 | 6.62 | 0.005 |
| minutes after | | | | |
| media change) | | | | |
| Day 5 | 6.75 | 0.02 | 6.43 | 0.019 |
| Day 6 | 6.79 | 0.01 | 6.21 | 0.010 |
| Day 7 (before | 6.79 | 0.005 | 6.17 | 0.003 |
| media change) | | | | |
| Day 7 (30 | 6.79 | 0.022 | 6.74 | 0.012 |
| minutes after | | | | |
| media change) | | | | |
| Day 8 | 6.81 | 0.012 | 6.56 | 0.009 |
| Day 9 | 6.75 | 0.022 | 6.37 | 0.019 |
| Day 10 | 6.69 | 0.027 | 6.21 | 0.015 |
| Day 11 (before | 6.65 | 0.026 | 6.18 | 0.005 |
| media change) | | | | |
| Day 11 (30 | 6.97 | 0.01 | 6.79 | 0.010 |
| minutes after | | | | |
| media change) | | | | |
| Day 12 | 6.85 | 0.014 | 6.73 | 0.008 |
| Day 13 | 6.81 | 0.003 | 6.70 | 0.007 |
| Day 14 | 6.78 | 0.015 | 6.62 | 0.036 |

The composition was then leached in 40 mL human osteoblast growth media in a humidified 37° C. incubator with 5% CO₂ for 24 hours while curing. After 24 hours of leaching, the composition was ready for plating. The media was aspirated from the composition in each well and the cured composition was cut using a razor blade into 1.5 cm pieces that were trimmed to fit into the wells of a 24 well plate.

Primary human osteoblast (HOB) cells were obtained from PromoCell GmbH (Heidelberg, Germany). Cultures were maintained with supplied HOB culture media according to the manufacturer instructions. Cells were seeded into the wells comprising the compositions at 15,000 cells/cm².

### Cell Viability

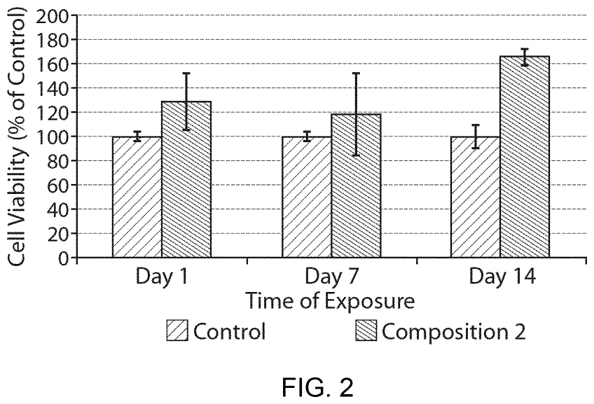

Cell viability was determined by measuring the reduction of 3-[4,5-dimethylthiazol-2-yl]-2,5-diphenyltetrazolium bromide (MTT, Sigma). The cells in each well were evaluated for their ability to reduce soluble MTT (yellow) to formazan-MTT (purple). An MTT stock solution was prepared in complete medium just prior to use and warmed to 37° C. in a water bath. Once the media was removed from all wells, MTT solution was added to each well and the plate was allowed to incubate at 37° C. for 3 hours. Media was removed and the purple formazan product was extracted using anhydrous isopropanol. Sample absorbance was read at 570 nm and reference absorbance at 650 nm with a Packard Fusion or equivalent plate reader. Cell viability and proliferation was determined by MTT uptake on days 1, 7, and 14. The results of this assay are summarized in Table 5 and FIG. 2.

TABLE 5
Cell viability (% of control) of osteoblasts exposed
to Composition 2 determined by MTT assay.

| | Osteoblasts | | |
|---|---|---|---|
| | Control | Composition 2 | |
| Time | (%) | (%) | p-value |
| Day 1 | 100 | 128.8 | 0.380 |
| Day 7 | 100 | 118.5 | 0.681 |
| Day 14 | 100 | 165.3 | 0.013 |

### Photomicrographs

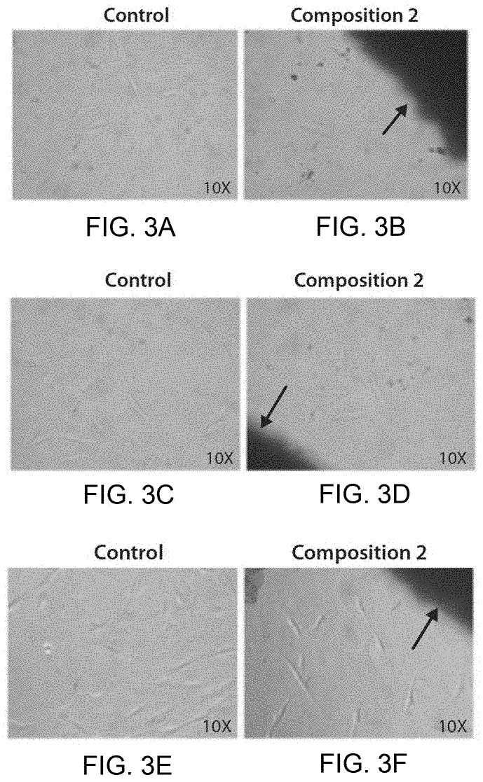

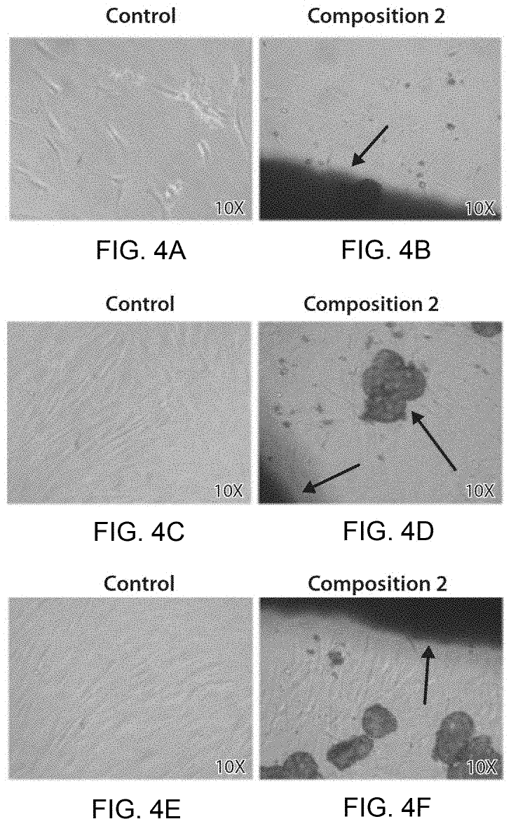

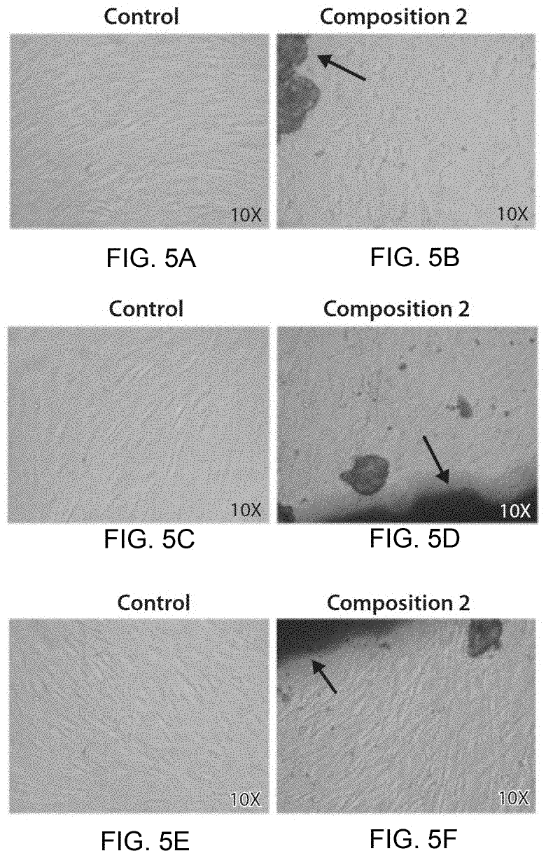

Photos were taken of cells by placing a Nikon CoolPix 4500 camera up to the ocular of a Nikon Eclipse TE200 microscope using 10× and/or 20× magnification. Photomicrographs were taken after 12 hours, 24 hours, then daily on days 3, 5, 7, 9, 11, 13, and 14 to assess morphology and cell density and are summarized in FIGS. 3A-3F, FIGS. 4A-4F, and FIGS. 5A-5F.

### Alkaline Phosphatase Activity

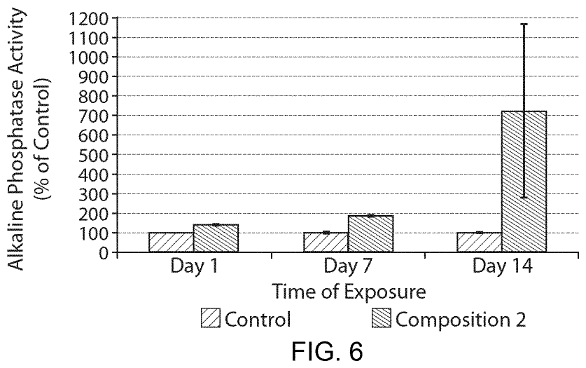

The change in alkaline phosphatase levels and activity is involved in a variety of physiological events, including bone development. The SensoLyte® FDP Alkaline Phosphatase Assay kit (AnaSpec) was used to determine the activity of alkaline phosphatase released into the media according to the manufacturer's instructions. 100 μL of each sample was transferred into a 96 well plate and fluorescence was measured by excitation at 485 nm with emission at 530 nm with a Packard Fusion or equivalent plate reader. Alkaline phosphatase activity was determined on days 1, 7, and 14. The results of this assay are summarized in Table 6 and FIG. 6.

TABLE 6
Alkaline phosphatase activity (% of control)
of osteoblasts exposed to Composition 2.

| | Osteoblasts | | |
|---|---|---|---|
| | Control | Composition 2 | |
| Time | (%) | (%) | p-value |
| Day 1 | 100 | 139.5 | 0.009 |
| Day 7 | 100 | 186.2 | 0.002 |
| Day 14 | 100 | 722.0 | 0.322 |

### Example 3: Analysis of In Vivo Biocompatibility of an Exemplary Composition

The objective of this study was to evaluate the local tissue effects, the performance (e.g., osteoinduction and bone ingrowth) and resorbability characteristics (e.g., degradation rate) of an exemplary composition.

### Study Design

Twelve male rabbits (New Zealand white) between the ages of 26-28 weeks old were obtained from Charles River Laboratories. Composition 2 was prepared as outlined in Example 2 using sterile conditions and implanted in a surgically created defect in the medial femoral condyle of both femurs in each rabbit (see experimental design in Table 7). For each femur, the target site was cancellous bone.

TABLE 7
Experimental design

| Subject No. | Time Period | |
|---|---|---|
| 1 | N/A | |
| 2 | 8 | weeks |
| 3 | | |
| 4 | | |
| 5 | 26 | weeks |
| 6 | | |
| 7 | | |
| 8 | | |
| 9 | 52 | weeks |

10
11
12

The rabbits were monitored for complete recovery following surgery and implantation. A subcutaneous injection of buprenorphine was administered at the end of surgery, then twice daily the day after surgery and once daily two days after the surgery. Additionally, subcutaneous injections of carprofen and enrofloxacin were administered daily for ten days after surgery. The overall health and behavior of the rabbits were monitored over the course of the study period.

At 8 weeks, 26 weeks, or 52 weeks, the rabbits were sacrificed and the site of implantation was visually inspected. The intact tissue envelope extending beyond the surgical area was removed, and femoral condyles and the inguinal lymph nodes were harvested for further analysis. All samples were fixed in 10% neutral formalin.

Histopathological analysis was conducted by digitalizing and examining fixed slides with a Zeiss Axioscope microscope equipped with a color image analyzing system. The percentage of bone ingrowth corresponding to bone area density, bone to implant contact, and biodegradation was calculated and statistically compared among the three time periods. The rate of degradation was theoretically evaluated at 26 and 52 weeks with respect to the previous time periods.

### Results

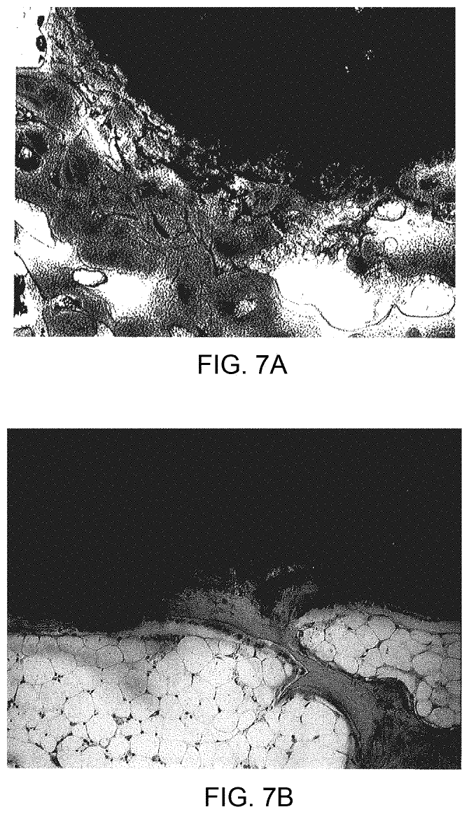

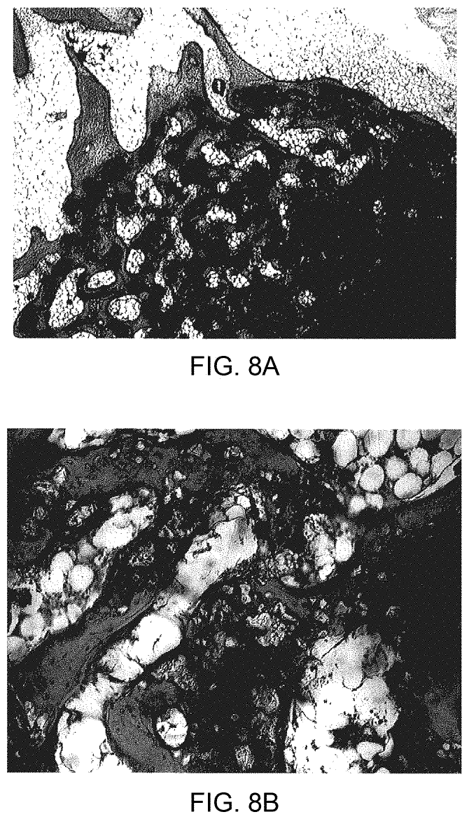

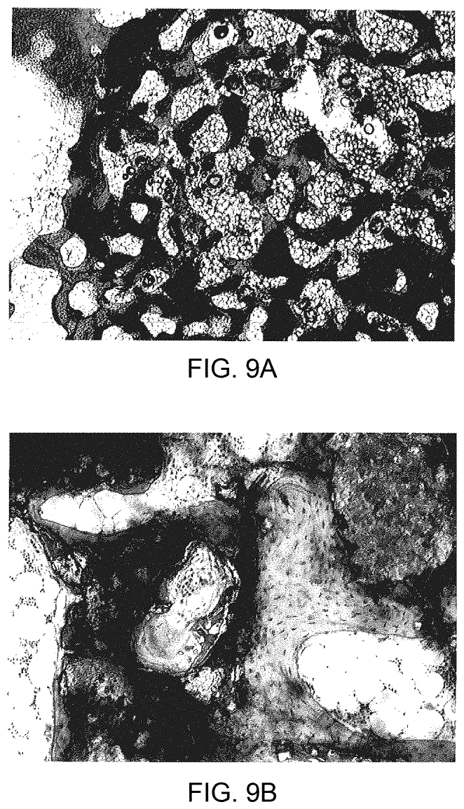

A total of 72 ground sections were analyzed. Some sections were excluded due to interference of the composition with the ligament, subchondral or cartilaginous tissues or fibrosis and border implantation. Representative photomicrographs were taken and are summarized in FIGS. 7A-7B (8 weeks), FIGS. 8A-8B (26 weeks) and FIGS. 9A-9B (52 weeks).

### 8 Week Time Period:

The composition was well maintained in situ and appeared a compact and homogenous material with numerous agglomerated particle as shown in FIGS. 7A-7B, with the section of the non-implanted material. Evidence of direct bone contact with the composition was observed in all sites. Some areas of the defect margin (smooth surface) in contact with the article did not show signs of bone repair. A moderate grade of newly formed bone apposition (osteoinduction) was observed. Bone ingrowth was frequently observed within the peripheral layer of the composition and in a limited number of slides, there was deep bone penetration (very slight grade) within the center of the composition. The bone remodeling process was graded slight. Numerous marginal thin bone debris (surgery-related) showing signs of osteonecrosis and resorption were observed around the implants and within the bone lacunae. The bone debris and particulate debris derived from the article were either osteointegrated or surrounded by inflammatory infiltrates. The inflammatory infiltrates were overall constituted of macrophages and multinucleated giant cell/osteoclasts graded moderate admixed with lymphocytes graded slight. In some slides, an extensive fibroinflammatory reaction diffusing up to 5 mm around the article and associated with slight signs of osteolysis were found. Signs of focal hypervascularization were observed in the presence of non-osteointegrated article particulate debris and fibroinflammatory reaction. Slight signs of composition degradation mediated by macrophages and multinucleated giant cells/osteoclasts were observed. No signs of cytotoxicity were observed.

### 26 Week Time Period:

The composition was moderately to markedly degraded resulting in a rough surface observed at the periphery of the implant. Signs of material degradation mediated by macrophages and multinucleated giant cells/osteoclasts were observed. Marked signs of osteoinduction and osteoconduction were observed. The bone conduction could be followed from one defect edge to another with an excellent bone to composition contact level. Normal bone marrow filled the newly formed bone lacunae taking place within the defect. The bone remodeling process was graded moderate. No residual bone debris (surgery-related) was observed around the implants. The inflammatory infiltrates diminished and were overall constituted of macrophages graded slight and multinucleated giant cells/osteoclasts and lymphocytes. In some slides, a focal fibroinflammatory reaction that could be associated with a cystic formation and slight signs of osteolysis was found. A few article particulate debris associated with macrophages were encountered around the implant. No evidence of cytotoxicity was observed. The 3 levels of section (4, 6, 8 mm) showed consistent results. Representative slides are shown in FIGS. 8A-8B.

### 52 Week Time Period:

The composition was markedly degraded resulting in small residual granules of the composition. Severe signs of osteoinduction and osteoconduction were observed. The granules were completely osteointegrated. The bone conduction could be followed from one defect edge to another with excellent bone to composition contact level. The bone trabecules remained thinner than normal trabecules reflecting an ongoing bone formation and remodeling process. The bone remodeling process was of a moderate grade and included the implant, recognized as a bone-like structure. Normal bone marrow filled the newly formed bone lacunae taking place within the defect. Haematopoetic bone marrow was formed. The composition degradation was mostly mediated through the remodeling process rather than through a macrophagic and multinucleated giant cell activity. The inflammatory infiltrates diminished and were overall constituted of macrophages and lymphocytes graded slight. No specific multinucleated giant cells were detected. No residual bone debris (surgery-related) was observed around the implants. In some slides, a focal fibroinflammatory reaction that could be associated with a cystic formation was found. No evidence of cytotoxicity was observed. The 3 levels of section (4, 6, 8 mm) showed consistent results. Representative slides are shown in FIGS. 9A-9B.

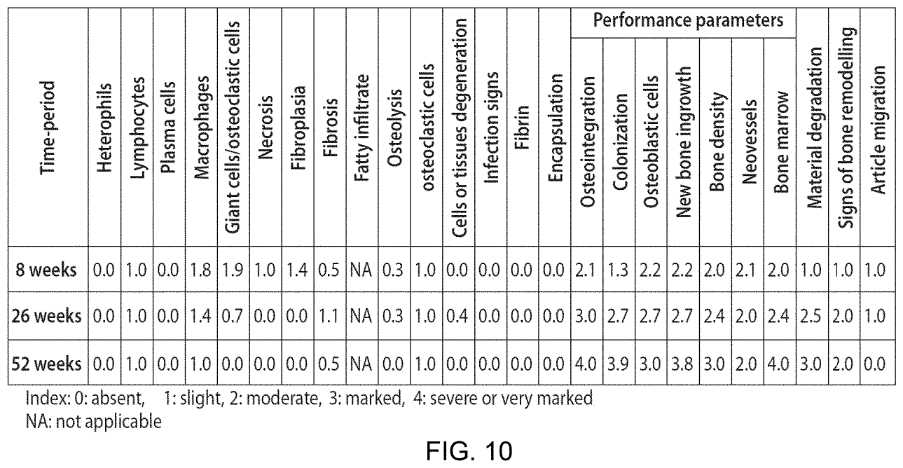

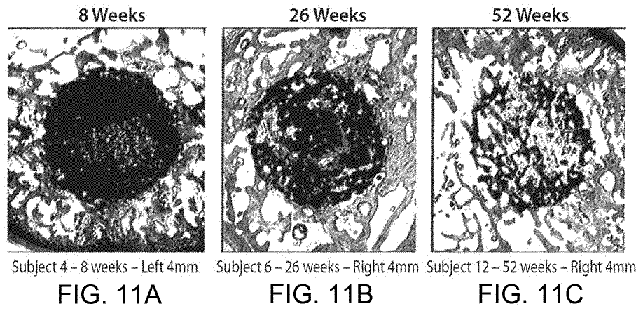

A table summarizing the semi-quantitative results of the histological analysis is shown in FIG. 10. FIGS. 11A-11C are representative photomicrographs from study subjects taken at 8 weeks, 26 weeks, and 52 weeks after implantation. Table 8 summarizes the percent degradation of composition 2 in the subjects over the course of the study.

TABLE 8
Summary of degradation of Composition 2.
Degradation percent (%) of Composition 2 over time

| | |
|---|---|
| Between 8 and 26 weeks | 48.8% |
| Between 26 and 52 weeks | 56.1% |
| Between 8 and 52 weeks | 77.5% |

### Example 4: Analysis of the In Vivo Local Tissue Effects of an Exemplary Composition

The objective of this study was to evaluate the local tissue effects (e.g., osteoinduction vs. bone loss) and biodegradation characteristics (e.g., dissolution, degradation, resorption kinetics) of Composition 3 in canine maxillae and mandibles as compared to controls.

Composition 3 consisted of 1625 mg total comprising the following powder components:

Tetra Calcium Phosphate, mean particle size=15-40 μm, 800 mg

O-Phospho-L-Serine, OPLS: 500 mg

Hydroxyapatite comprising granules, granule Ø=0.100-0.250 mm; 70% porosity, pore Ø=10-130 μm, 325 mg

The powder components of the composition were individually weighed into glass vials sealed with rubber stoppers. The plastic caps were crimped and the vials were gamma irradiated (15-25 kGy) prior to testing.

Composition 3 was mixed with 325 μL sterile H₂O in a 25 mL silicone mixing bowl to create a homogeneous viscous fluid which was deposited into the barrel of a 3 cc syringe for application.

### Study Design

Three skeletally mature canines (12 to 15 months of age) were selected as subjects for this study. Eight sites were developed per canine for a total of 12 maxillary and 12 mandibular sites under general anesthesia. The sites for the implantation were prepared at locations indicated in Table 9. The procedure consisted of extraction of the indicated premolar teeth and resection of the alveolar ridge at locations indicated from the midridge buccally and from the crest apically to the apices of the extracted teeth or 8 mm, whichever was lesser. The bone was allowed to heal for seven weeks undisturbed before the sites were to be used for implantation of test articles or controls.

Composition 3 was injected under general anesthesia into the bony defects uncovered by reflection full thickness flaps. The composition was allowed to cure prior to closure of the sites with resorbable sutures.

TABLE 9
Experimental Design, Summary of Anatomical Locations Used as
Implantations Sites for Composition 3 in the Jaws of Canines

| | | |
|---|---|---|
| | Maxilla | |
| R | Incisors (x6) | L |

| | | | |
|---|---|---|---|
| Cx | | | Cx |
| P1 | 2 | 3 | P1 |
| P2m | | | P2m |
| P2d | | | P2d |
| P3m | 1 | 4 | P3m |
| P3d | | | P3d |
| P4m | | | P4m |
| P4d | | | P4d |
| M1 | | | M1 |
| M2 | | | M2 |

Mandible

| | | | |
|---|---|---|---|
| R | | | L |
| M3 | | | M3 |
| M2 | | | M2 |
| M1 | | | M1 |
| P4d | 8 | 5 | P4d |
| P4m | | | P4m |
| P3d | | | P3d |
| P3m | | | P3m |
| P2d | 7 | 6 | P2d |
| P2m | | | P2m |
| P1 | | | P1 |
| Cd | | | Cd |

Incisors (x6)

### Results

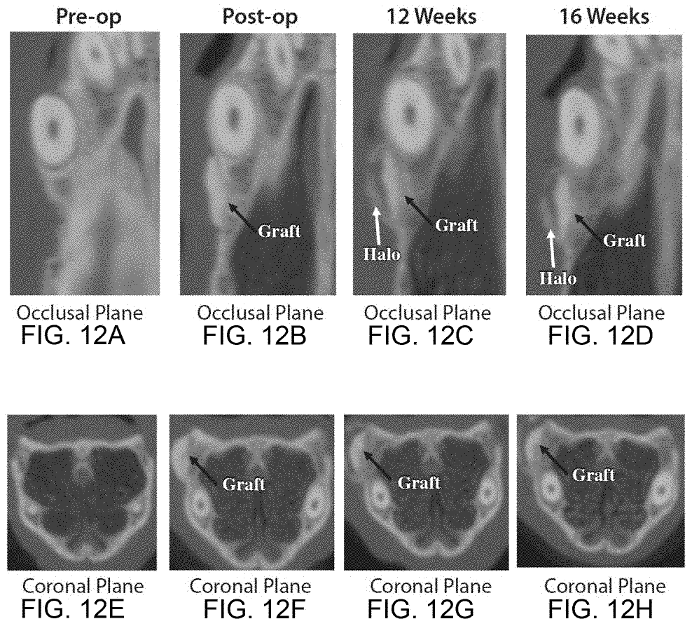

The progress of the presurgical, surgical and postoperative changes was recorded through the use of Cone Beam Computed Tomography (CBCT) scans. FIGS. 12A-12H demonstrate the changes occurring at a particular site (Site #2, maxillary right anterior site) in a particular subject animal (Canine C5) during the course of the study from immediately prior to the implantation of Composition 3 to 16 weeks post-operative. Note the dimensions of the bony structures preoperatively as compared to the size of the Composition 3 graft deposit. As shown in FIGS. 12A-12H, no significant bone loss occurred in the vicinity of the Composition 3 graft deposit. At sixteen weeks, new bone formation can be seen as a “halo” developing circumferentially in the proximity of the deposition site of Composition 3. Histological examination of the tissue from another subject animal (Example 5) confirms the new bone deposition, indicating the osteoinductive character of the composition.

### Example 5: Evaluation of the In Vivo Local Tissue Effects of an Exemplary Composition

The objective of this study was to evaluate the local tissue effects (e.g., osteoinduction vs bone loss, or excessive or continuing inflammation) and biodegradation characteristics (e.g., dissolution, degradation, resorption kinetics) of Composition 3 in canine maxillae and mandibles as compared to controls.

### Study Design

Five skeletally mature canines (12 to 13 months of age) weighing at least 12 kg were included in the study. The sites for the onlay graft implantation of the test compositions were located as indicated in Table 10. Four onlay sites were available per animal: two maxillary and two mandibular. All the sites were located on the buccal aspect of the maxilla or the mandible, immediately overlying the cuspid root, and were subperiosteal.

The implantation procedure consisted of gaining access to the sites under general anesthesia through a full thickness tunneling approach from a vertical incision located at least 10 mm anterior to the deposition locus. Composition 3 was prepared as outlined in Example 4 using sterile conditions. At least 0.5 cc of the composition was then deposited by injection into the tunnel and directly onto the bone surface. The compositions were left to cure for three minutes before the access to the deposited material in the pocket was closed with resorbable sutures. The animals were followed for different lengths of time before they were sacrificed and the tissues examined. The subject animal presented in this example (Canine C4) was sacrificed 10 weeks post-implantation. The site presented in this example is the mandibular right site (#4).

TABLE 10
Experimental Design, Summary of Anatomical Locations Used as
Implantations Sites for Composition 3 in the Jaws of Canines

| | R | | | | | | | L | |
|---|---|---|---|---|---|---|---|---|---|
| | | Maxilla | | | | | | | |
| Arm | | | | | | | | | Arm |
| 1 | | Ix-3 | Ix-2 | Ix-1 | Ix-1 | Ix-2 | Ix-3 | | 2 |
| | Cx | | | | | | | Cx | |
| | Px-1 | | | | | | | Px-1 | |
| | Px-2m | | | | | | | Px-2m | |
| | Px-2d | | | | | | | Px-2d | |
| | Px-3m | | | | | | | Px-3m | |
| | Px-3d | | | | | | | Px-3d | |
| | Px-4m | | | | | | | Px-4m | |
| | Px-4d | | | | | | | Px-4d | |
| | Mx-1 | | | | | | | Mx-1 | |
| | Mx-2 | | | | | | | Mx-2 | |
| | R | | | | | | | L | |
| | | Mandible | | | | | | | |
| Arm | | | | | | | | | Arm |
| | Md-3 | | | | | | | Md-3 | |
| | Md-2 | | | | | | | Md-2 | |
| | Md-1 | | | | | | | Md-1 | |
| | Pd-4d | | | | | | | Pd-4d | |
| | Pd-4m | | | | | | | Pd-4m | |
| | Pd-3d | | | | | | | Pd-3d | |
| | Pd-3m | | | | | | | Pd-3m | |
| | Pd-2d | | | | | | | Pd-2d | |
| | Pd-2m | | | | | | | Pd-2m | |
| | Pd-1 | | | | | | | Pd-1 | |
| 4 | Cd | | | | | | | Cd | 3 |
| | | Id-3 | Id-2 | Id-1 | Id-1 | Id-2 | Id-3 | | |

### Results

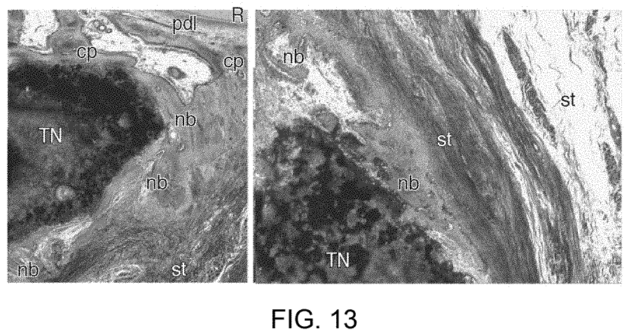

The tissues obtained from the implantation sites were fixed in alcohol, mounted in plastic and stained with Masson's Trichrome for histological examination. The image presented in FIG. 13 is that of a photomicrograph recorded from the site #4, the onlay graft comprising Composition 3 adhered to the facial aspect of canine mandible at cuspid root level at ten weeks post-implantation (unmineralized ground section). The cuspid root (R), periodontal ligament (pdl), original limit of the bone, the cortical plate (cp), Composition 3 deposit mass (TN), new bone (nb), and the labial soft tissues (st) are marked for orientation. Note the integration of the graft mass with the maxillary bone and the deposition of new bone (nb) both circumferentially at the junction between the graft and bone and superficially to the graft facing the soft tissues. Note the absence of inflammatory infiltrate and the presence of new woven bone covering the surface of the Composition 3 deposit. At ten weeks, new bone formation can be seen as a “halo” developing circumferentially in the proximity of the deposition site of Composition 3, indicating its osteoinductive character. Radiographic examination of the tissue from another subject animal at week 16 (see Example 4) confirms the new growth as bone based in part on its radiodense character.

### INCORPORATION

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A method of generating or regenerating bone tissue, the method comprising:
a) preparing a composition comprising a multivalent metal salt and an osteoinductive factor in an aqueous solution or suspension;
b) applying the composition to a site (e.g., into or onto bone, or in between bones); and
c) allowing the composition to harden, cure, or be resorbed by bone;
wherein the osteoinductive factor is a compound of Formula (I): 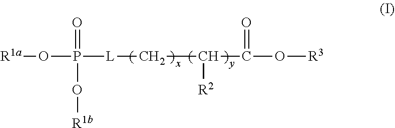
or a pharmaceutically acceptable salt thereof, wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4a} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3; and
the multivalent metal salt comprises calcium.

2. The method of claim 1, wherein the generation or regeneration is derived from the increased action of osteoblast cells.

3. The method of claim 1, wherein the generation or regeneration further comprises an increase in alkaline phosphatase activity, e.g., relative to a reference standard.

4. A method of inducing osteoblast formation, the method comprising:
a) preparing a composition comprising a multivalent metal salt and an osteoinductive factor in an aqueous solution or suspension;
b) applying the composition to a site (e.g., into or onto bone, or in between bones); and
c) allowing the composition to harden, cure, or be resorbed by bone;
wherein the osteoinductive factor is a compound of Formula (I): 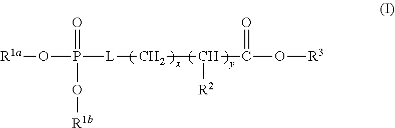
or a pharmaceutically acceptable salt thereof, wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4a} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3; and
the multivalent metal salt comprises calcium.

5. The method of claim 4, wherein the formation of osteoblasts is derived from the differentiation of mesenchymal stem cells.

6. A method of treating or preventing a bone disease or disorder in a subject, the method comprising:
a) preparing a composition comprising a multivalent metal salt and an osteoinductive factor in an aqueous solution or suspension;
b) applying the composition to a site (e.g., into or onto bone, or in between bones); and
c) allowing the composition to harden, cure, or be resorbed by bone;
wherein the osteoinductive factor is a compound of Formula (I): 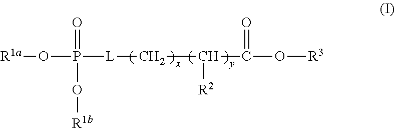
or a pharmaceutically acceptable salt thereof, wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4a} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3; and
the multivalent metal salt comprises calcium.

7. The method of claim 6, wherein the bone disease or disorder comprises cancer (e.g., osteosarcoma), osteoporosis, rickets, osteogenesis imperfecta, Paget's disease of the bone, hearing loss, renal osteodystrophy, a malignancy of the bone, infection of the bone, osteonecrosis, or other genetic or developmental disease.

8. The method of claim 1, wherein the multivalent metal salt comprises tetracalcium phosphate.

9. The method of claim 1, wherein the composition comprises tetracalcium phosphate and at least one other multivalent calcium compound.

10-16. (canceled)

17. The method of claim 1, wherein the compound of Formula (I) is phosphoserine.

18. The method of claim 1, wherein the osteoinductive factor (e.g., a compound of Formula (I)) is present in an amount greater than or equal to about 10% (w/w) of the total composition.

19. The method of claim 1, wherein the aqueous solution or suspension comprises water, saliva, saline, serum, plasma, or blood.

20. The method of claim 1, wherein the multivalent metal salt is initially provided as granules or a powder.

21. The method of claim 1, the composition further comprises an additive.

22. The method of claim 1, wherein the method further comprises release of the osteoinductive factor from the composition.

23. The method of claim 22, wherein the release of the osteoinductive factor takes place over the course of minutes, hours, days, months, or years.

24. The method of claim 1, wherein the generation or regeneration of bone tissue or the formation of osteoblasts is correlated with an increase in the levels of a biomarker relative to a reference standard.

25. The method of claim 24, where the biomarker comprises alkaline phosphatase, osteocalcin, matrix gla protein, or osteopontin, or collagen (e.g., type I collagen).

26. A kit for use in the generation or regeneration of bone tissue, wherein the kit comprises:
(a) an osteoinductive factor comprising a compound of Formula (I): 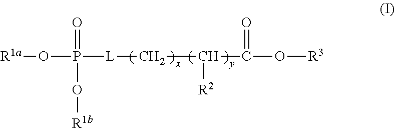
or a pharmaceutically acceptable salt thereof, wherein:
L is O, S, NH, or CH₂;
each of R^{1a} and R^{1b} is independently H, optionally substituted alkyl, or optionally substituted aryl;
R² is H, NR^{4a}R^{4b}, C(O)R⁵, or C(O)OR⁵;
R³ is H, optionally substituted alkyl, or optionally substituted aryl;
each of R^{4a} and R^{4a} is independently H, C(O)R⁶, or optionally substituted alkyl;
R⁵ is H, optionally substituted alkyl, or optionally substituted aryl;
R⁶ is optionally substituted alkyl or optionally substituted aryl; and
each of x and y is independently 0, 1, 2, or 3;
(b) a multivalent metal salt comprising calcium;
(c) an aqueous medium; and optionally,
(d) an additive (e.g., biologically active substance),
wherein each of (a), (b), (c), and (d) is contained within a separate container.

27. The method of claim 4, wherein the compound of Formula (I) is phosphoserine.

28. The method of claim 6, wherein the compound of Formula (I) is phosphoserine.

29. The method of claim 4, wherein the osteoinductive factor (e.g., a compound of Formula (I)) is present in an amount greater than or equal to about 10% (w/w) of the total composition.

30. The method of claim 6, wherein the osteoinductive factor (e.g., a compound of Formula (I)) is present in an amount greater than or equal to about 10% (w/w) of the total composition.

31. The method of claim 4, the composition further comprises an additive.

32. The method of claim 6, the composition further comprises an additive.

33. The method of claim 4, wherein the method further comprises release of the osteoinductive factor from the composition.

34. The method of claim 33, wherein the release of the osteoinductive factor takes place over the course of minutes, hours, days, months, or years.

35. The method of claim 6, wherein the method further comprises release of the osteoinductive factor from the composition.

36. The method of claim 35, wherein the release of the osteoinductive factor takes place over the course of minutes, hours, days, months, or years.
